(19) United States
(12) Patent Application Publication
(10) Pub. No.: US 2006/0167401 A1
(43) Pub. Date: Jul. 27, 2006

(54) Apparatus and method for stem cell preservation and usage

(75) Inventor: Michael Cohen, West Orange, NJ (US)

Correspondence Address: PEARL COHEN ZEDEK, LLP, 1500 BROADWAY 12TH FLOOR, NEW YORK, NY, 10036, US

(73) Assignee: National Stem Cell Inc

(21) Appl. No.: 11/038,119

(22) Filed: Jan. 21, 2005

(51) Int. Cl. A61M 37/00

(52) U.S. Cl. 604/6.15

(57) ABSTRACT

An apparatus and method may provide effective preservation and usage of preserved blood or blood components. A blood preservation apparatus may include a plurality of collection bags that may be separated by separator mechanisms to enable rapid separation of the collection bags from the blood preservation apparatus when the apparatus is frozen.

### FIELD OF THE INVENTION

The present invention relates to methods and devices useful in preservation and usage of blood or blood components, or other biological matter. Specifically, embodiments of the present invention relate to systems, methods and apparatuses that enable effective preservation and usage of components such as stem cells.

### BACKGROUND OF THE INVENTION

Umbilical cord blood, which is the blood left in the umbilical cord and placenta after birth, has recently been recognized as a life-saving substance since umbilical cord blood, like bone marrow, contains regenerative “stem” cells. Stem cells are the primitive cells from which other blood cells develop. Thus, they are the building blocks of the immune and blood cell systems. Cord blood stem cells are unique in that they are immature “naive” immune cells. When they are used in transplantation, they have reduced reactivity and are less likely to recognize the recipient as foreign. When transplanted into a sick person, even a single stem cell has the ability to regenerate the person's blood system. For this reason, stem cells are vital when a person's immune system has been weakened by radiation or chemotherapy, or by diseases that attack the immune system. Stem cells are highly effective against many diseases.

Cord blood may be collected from the umbilical cord immediately after the birth of the baby but generally before the placenta has been delivered. The collection is generally done at the time of delivery, and must be stored at temperatures of approximately −100 degrees Celsius (100° Celsius below zero) or less to maintain the stem cells intact for future usage. The amount of cord blood that can be collected is therefore limited, and it is important for people wishing to collect and store core blood that a maximum amount of core blood is collected and appropriately stored. Once stored core blood is removed from its storage container and its temperature increases to above approximately −91 Celsius (91° Celsius below zero) the stem cells may no longer be stored for later usage.

Stem cells may also be attained from bone marrow. Since the potential of the stem cells in bone marrow has been recognized, many deaths per year are being prevented by bone marrow transplants. However, bone marrow is in short supply. Additionally, since cord blood also contains regenerative “stem” cells, it is being used as preferable alternative to bone marrow, and the number of cord blood transplants has increased significantly during recent years. Many more lives could be saved if umbilical cord blood were routinely banked and its additional stem cells made available for transplants. Adults as well as children have been successfully treated with cord blood, and cord blood can be cryo-preserved in liquid nitrogen and remain usable for years. If a child's cord blood is preserved in this way, the grown individual can use his or her own stem cells later in life to fight various serious diseases.

Collection of cord blood has typically been accomplished by attempting to direct a cord blood stream into opened laboratory test tubes and then capping the test tubes. Usually, spillage of cord blood occurs during this procedure, which is difficult for one person to perform. The blood spillage is doubly unfortunate because it not only wastes the precious blood but also increases the health workers' exposure to a potential HIV or other hazards. To decrease the amount of spilled blood, a funnel or other device might be provided. However, to support a funnel and a test tube having a stopper while dripping blood would be even more awkward than merely filling a test tube. The used funnel would be another piece of contaminated trash. The test tube also ends up as contaminated trash and, if made of glass, it presents a puncture hazard in the event of breakage.

After a test tube or test tubes are filled with the desired amounts of blood and taken away, the attending pediatricians frequently call for a laboratory technician to come to the delivery room or newborn nursery soon after the birth to perform additional venipunctures or heel sticks on the newborn infant to obtain more blood samples for base line studies about the condition of the infant These additional intrusions could of course be avoided by performing the same tests on cord blood, if more were available. However, the test tubes are gone by that time.

To collect larger amounts of cord blood for later tests or for long term preservation, the primitive and messy method of dripping blood into test tubes is relatively inefficient. At the present time bulk umbilical cord blood collection is performed mainly by using 50 cc syringes that contain a heparinized solution. The syringes are sent to pregnant women in the form of a kit. The expectant mother or parents are then responsible for bringing the kit to their delivery. The syringes, with needles attached, are given to the delivering doctor.

After delivery of the infant and cutting and clamping the cord the physician may insert the needle into the distal umbilical cord vein and slowly aspirate available cord blood into the syringes. This method requires a relatively long time (about five minutes) and a substantial amount of effort. Several syringes may be used (each being capped, labeled, etc.) and the needles must be inserted at numerous places along the cord for best results; each insertion site must be swabbed prior to sticking with the needle. The average amount collected is around 80 cc.

The yield may be low because, first, blood is drawn only from the single umbilical cord vein, and not from either of the two arteries, and second, because the collection efficiency of applied suction is low. Besides the large amounts of time and effort required, and the low yield, this method of collecting cord blood may be dangerous because of the several needles that need to be uncovered, used, removed, and disposed of, and the because of the numerous sticks into the thin-walled umbilical vein of the slippery cord. The syringes, tray, and needles constitute a great deal of hazardous waste material and/or extra cleaning and sterilizing work.

### SUMMARY OF THE INVENTION

There is provided, in accordance with an embodiment of the present invention, an apparatus, system, and method for preserving blood and/or blood components, and in particular, stem cells. According to some embodiments of the present invention, a blood preservation bag that includes multiple compartments separated by separator mechanisms may be used to store blood. The compartments may be easily separated from one another while the bags are still frozen, thereby enabling usage of a part of the blood supply stored in the bags, while keeping the remaining blood supply viable for future use. In one embodiment the separator mechanisms include a perforated line. The connectors may be joined to a bag such that a minimal amount of pressure is placed on the bag wall, to prevent unwanted tearing of the bag wall.

Moreover, in accordance with an embodiment of the present invention, the blood preservation apparatus may include one or more blood entry tubes, which may include a heat-sealed area, and/or a valve. In some embodiments a flexible or foldable joint may be provided in the blood entry tubes to enable an entry tube to be folded towards a blood bag, to prevent unintentional breaking off of the entry tube, thereby enabling usage of the tube, for example, for DNA testing. In accordance with some embodiments of the present invention, a holding strap may be provided to connect tubes to bags. In another embodiment the individual bags may be angled so as to enable a maximum amount of bag content to flow out during usage. In some embodiments the blood preservation apparatus may include a blood distribution tube, which may include a diverter mechanism.

According to some embodiments of the present invention, a blood preservation method is provided that includes collecting stem cells in a blood preservation apparatus, the apparatus having a plurality of blood collection containers being separated by separator mechanisms that enable separation of at least one blood collection bag from the blood preservation apparatus while the blood preservation apparatus is substantially frozen; and freezing the blood preservation apparatus. The method may further include separating a blood collection bag while non-required elements of the blood preservation apparatus remain substantially frozen. Further, the method may include returning the blood preservation apparatus to a blood freezing source before the contents of the blood preservation apparatus have substantially thawed. In one embodiments the contents extracted from the separated portion may be expanded.

In one embodiment of the present invention a blood preservation apparatus is provided that includes a plurality of blood entry tubes and separator mechanisms, the separator mechanisms to enable at least one the blood entry tubes to be separated from the blood preservation apparatus while the blood preservation apparatus is substantially frozen.

### BRIEF DESCRIPTION OF THE DRAWINGS

The principles and operation of the system, apparatus, and method according to the present invention may be better understood with reference to the drawings, and the following description, it being understood that these drawings are given for illustrative purposes only and are not meant to be limiting, wherein:

FIG. 1 is a schematic drawing of an embodiment of a blood bag apparatus, according to some embodiments of the present invention; and

FIG. 2 is a flowchart illustrating a method for collecting blood, according to some embodiments of the present invention.

It will be appreciated that for simplicity and clarity of illustration, elements shown in the drawings have not necessarily been drawn to scale. For example, the dimensions of some of the elements may be exaggerated relative to other elements for clarity. Further, where considered appropriate, reference numerals may be repeated among the drawings to indicate corresponding or analogous elements throughout the serial views.

### DETAILED DESCRIPTION OF THE INVENTION

The following description is presented to enable one of ordinary skill in the art to make and use the invention as provided in the context of a particular application and its requirements. Various modifications to the described embodiments will be apparent to those with skill in the art, and the general principles defined herein may be applied to other embodiments. Therefore, the present invention is not intended to be limited to the particular embodiments shown and described, but is to be accorded the widest scope consistent with the principles and novel features herein disclosed. In other instances, well-known methods, procedures, and components have not been described in detail so as not to obscure the present invention.

The word “blood” as used herein may encompass stem cells, bone marrow, plasma, red blood cells, white blood cells, and/or platelets or other elements or components of mammalian blood cells. The word “preservation” as used herein may encompass storing, freezing or otherwise preserving stored blood, blood cells, and/or platelets or other elements of mammalian blood cells. The word “usage” as used herein may encompass thawing, testing, expanding, transplanting or otherwise utilizing stored blood, blood cells, and/or platelets or other elements of mammalian blood cells. An apparatus for preservation and usage of blood may be applied to blood or other suitable bodily fluids.

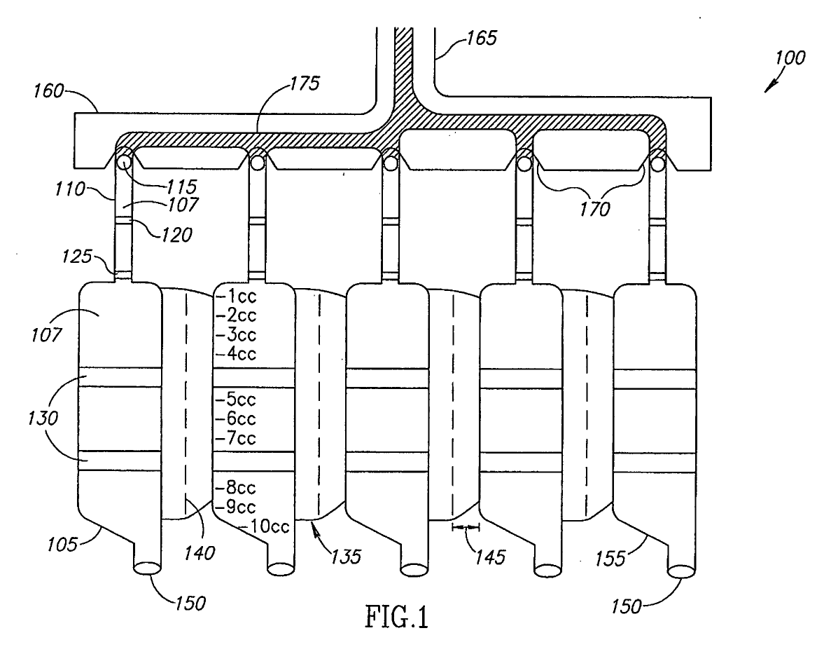

Reference is now made to FIG. 1, which is a schematic drawing illustration of a blood collection, preservation and usage apparatus 100, herein referred to as “blood collection apparatus” or “apparatus”, according to some embodiments of the present invention. Apparatus 100 may include a multi-part collection bag that may include a plurality of connected blood collection containers, for example, including one or more collection bags 105, blood entry tubes 110, and other suitable blood holding containers that may be rapidly separated from apparatus 100. Bags 105 may include blood entry tubes 110 through which blood may enter bag 105. Blood may enter into tube 110 through valve 115, which may be a one-way valve such that no blood may flow out of valve 115. Tube 110 may have a joint, for example, foldable joint 125 substantially adjacent to the lower part of tube 110. Foldable joint 125 may enable tube 110 to be folded over, for example, to lie parallel to bag 105, to minimize the risk of tube 110 being damaged or severed etc. from bag 105, and/or to reduce storage space of apparatus 100. Bag 105 may include one or more straps 130. Strap 130 may be used, for example, to hold tube 110 when tube 110 is folded over towards bag 105, to stabilize or otherwise secure tube 110 to bag 105. Straps 130 may be used to secure bags 105, and/or other suitable elements of apparatus 100.

Tube 110 may have a separator element 120, which may include, for example, a heat seal or other suitable seal to lock or seal contents in bag 105. For example, tube 110 may be heat sealed at location 120 and/or other suitable locations, to enable the formation of two or more parts of tube 110. In this way part of the contents of tube 110 may be separated and utilized, and the remaining parts of tube 110 may optionally be further preserved and utilized on separate occasions. For example, if tube 110 holds 3 cc of blood, this may be divided into two parts holding, for example, 1 cc and 2 cc. If, for example, a DNA test is required to determine compatibility of the bag contents to a donors blood, 2 cc may be required, in which case the 2 cc portion may be separated from the 1 cc portion, and utilized for testing etc., while keeping the 1 cc portion frozen and available for future usage. Other amounts of blood may be preserved in one or more compartments or sub-sections of bag 105, tube 110, and/or other elements of apparatus 100.

Elements of apparatus 100 may be constructed from commonly used blood bag material. For example, bag 105 may be constructed from polytetrafluoroethylene fabric or other suitable fabrics or combinations of fabrics. In one embodiment bag 105, for example a cryopreservation bag, may include a body made of two sheets of polyamide/fluoropolymer laminate film with a joining piece sealed between these sheets and a section of non-PVC tubing assembled on the joining piece, the assembly being strengthened and tightened with a shrink tube. The cryopreservation bag may be equipped with a tubing that is resistant to very low temperatures and that is sealable and sterile dockable both prior to and after being subjected to liquid nitrogen, thereby providing the means for a safe transfer of blood to and from the bag and helping prevent contamination. One example for the composition of such tubing is a blend of Hytrel.COPYRGT. (DuPont de Nemours) and Estane.COPYRGT. (B.F. Goodrich Chemical Co.), chemically known as thermoplastic polyester elastomer and thermoplastic polyurethane elastomer, in a ratio of respectively 80% and 20% Other suitable materials or combinations of materials may be used.

Bag 105 may have a separator 135 that may connect two or more bags 105. Separator 135 may include a separation element, for example, a perforation line 140, zipper, button, knot, Velcro™ hook and loop connector or other hook and loop connector, adhesive or other suitable mechanism to enable two or more bags 105 or sub-sections of apparatus 100 to be connected in such a way as to allow rapid and easy separation, for example, under freezing conditions. Separator 135 may connect two or more bags or other components such that a second bag or component is partially connected to separator 135. For example, a gap indicated by 145 may be left between separator 135 and a second bag 105 to limit or prevent unwanted tearing or damage to second bag 105.

Bag 105 may have an exit point 150 through which the contents may be extracted or released from bag 105. Bag 105 may have a non-flat or downward slanted lower wall 155 to enable bag content to flow out of exit point 150 using gravity, to help rapidly empty bag 105 of substantially all its contents when unfrozen.

Bags 105 and/or blood entry tubes 110 may be filled with blood 107 via, for example blood entry tubes 110. One or more tubes 110 may be connected to a blood distribution tube or feed tube 160, which may be connected by connection tube 165 to a blood source (e.g., umbilical cord, blood testing or filtering machine, blood extraction mechanism etc.). Distribution tube 160 may be made from the same material as connection tube 165. In one example, connection tube 165 is the distribution tube. Blood may enter connection tube 165 from any suitable open ended blood processing system. The configuration of the connector of 165 to other systems may be varied. Blood distribution tube 160 may include a blood diverter mechanism 175 and one or more blood exit points 170 to enable distribution of blood into one or more bags 105. In one embodiment blood diverter mechanism 175 may enable a first bag 105 to be filled with blood, followed by a first tube 110. After a first bag 105 and tube 110 have been filled, a second bag 105 and tube 110 may be filled, etc., using blood diverter mechanism 175. In one embodiment blood diverter mechanism 175 may include an on/off switch, which for example may be manually operated to allow for blood flow in one direction only. Since the switch does not necessarily have to endure the cryopreservation process, the switch may be composed of various suitable materials, for example, PVC or other suitable materials. Any number of bags 105, tubes 110, valves 115, joints 125, exit points 150, separators 135, blood distribution tubes 160, blood diverter mechanisms 175 and blood exit points 170 etc. may be used. For example, in some embodiments 5 bags 105 may be used, wherein each may 105 may hold 10 cc of blood, and each tube 110 may hold 3 cc of blood. Other structures and dimensions, holding other amounts of blood may be used, for example, compartments may hold 50 cc or other selected volumes of blood.

In some embodiments of the present invention, blood preservation apparatus 100 may include a plurality of blood entry tubes and separator mechanisms, wherein the separator mechanisms may enable one or more blood entry tubes to be separated from the apparatus while the apparatus is substantially frozen.

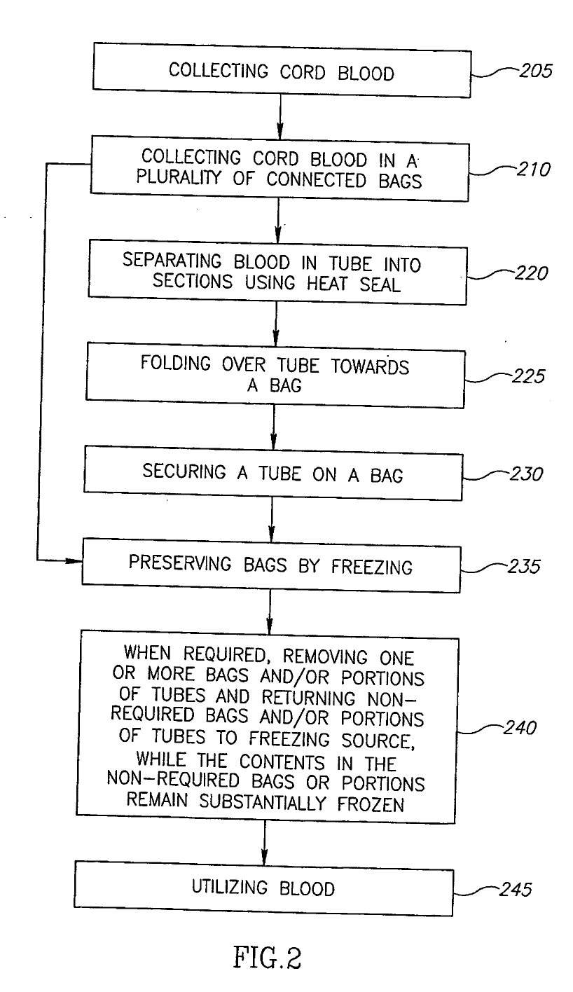

FIG. 2 schematically illustrates a series of operations or processes that may be implemented, according to some embodiments of the present invention. While the apparatus such as that shown in FIG. 1 may be used, other suitable apparatuses may be used. As can be seen in FIG. 2, at block 205 blood may be collected from a suitable blood source, for example an umbilical cord, placenta, bone marrow source etc. At block 210 the blood may enter or be collected in a blood bag such as bag 105, via a blood entry tube 110. Preferably when bag 105 and tube 110 are substantially full, blood may enter into further bags via further blood entry tubes, as described above. At block 220 tube 110 may be separated into two or more sections, for example, using a heat seal. At block 225, tube 110 may be folded over towards bag 105, at foldable joint 125. At block 230 tube 110 may be secured to bag 105, for example, using strap 130. One or more tubes and/or containers may be secured to apparatus 100 using at least one strap 130.

At block 235, blood collection, preservation and usage apparatus 100 may be preserved, for example, by freezing apparatus 100 in liquid Nitrogen or in other suitable ways. Blood including stem cells may generally be preserved for future usage by being stored between −100 to −120° Celsius. Blood should be stored at such temperatures since when the blood temperature drops beneath approximately −91° Celsius, for example, which may take approximately 1 minute, many of the stem cells may be corrupted or destroyed, and the blood may not be refrozen.

At block 240 at least one portion of the preserved blood may be used, for example, by removing apparatus 100 from the storage facility and rapidly removing one or more bags 105, portions of tube 110 and/or other sub-sections or portions of apparatus 100, and returning the remainder of apparatus 100 to the storage facility while the non-required contents in apparatus 100 remain substantially frozen at a suitable temperature, for example, below −91° Celsius. For example, one or more bags, tubes, or parts of bags or tubes may be separated from the apparatus by using a guillotine, a ripping motion, a scissors, a hand, or other suitable equipment or methods to rapidly separate at least part of the bags or tubes from apparatus 100. At block 245 the contents from the bag(s) and/or tube(s) separated from apparatus 100 may be extracted and utilized.

In one embodiment of the present invention the contents from the bag(s) and/or tube(s) separated from apparatus 100 may be expanded, for example, using blood expansion techniques. For example, 10 cc of umbilical cord blood may be expanded using Gamida-Cell stem/progenitor cell expansion technology (which may be provided by Gamida-Cell Inc., New York, N.Y. 10011 USA) or Pluristem Life Systems Inc. technology (Haifa, Israel), to provide sufficient stem cells for appropriate treatments to children and/or adults. Any combination of the above operations may be implemented. Further, other steps or series of operations may be used.

The foregoing description of the embodiments of the invention has been presented for the purposes of illustration and description. It is not intended to be exhaustive or to limit the invention to the precise form disclosed. It should be appreciated by persons skilled in the art that many modifications, variations, substitutions, changes, and equivalents are possible in light of the above teaching. It is, therefore, to be understood that the appended claims are intended to cover all such modifications and changes as fall within the true spirit of the invention.

1. A blood preservation apparatus, comprising a plurality of blood collection containers and at least one separator mechanism, said separator mechanism to enable at least one blood collection container to be separated from the blood preservation apparatus while the blood preservation apparatus is substantially frozen.

2. The apparatus of claim 1, wherein said blood collection containers are blood bags.

3. The apparatus of claim 1, wherein said blood collection containers are blood entry tubes.

4. The apparatus of claim 3, wherein said blood entry tubes include at least a foldable joint.

5. The apparatus of claim 3, wherein said blood entry tubes include at least a heat-seal.

6. The apparatus of claim 3, wherein said blood entry tubes include at least a valve.

7. The apparatus of claim 3, wherein said blood entry tubes include at least an on-off switch mechanism.

8. The apparatus of claim 1, further comprising a blood distribution tube.

9. The apparatus of claim 8, wherein said blood distribution tube includes a diverter mechanism.

10. The apparatus of claim 1, wherein said separator mechanism includes a mechanism selected from the group consisting of a perforation line, zipper, button, knot, hook and loop connector and adhesive.

11. The apparatus of claim 1, wherein the lower wall of said collection container is angled to enable the container contents to be released from the container by gravity.

12. The apparatus of claim 1, comprising a strap to secure one or more blood preservation apparatus components.

13. The apparatus of claim 12, further comprising a blood entry tube, said blood entry tube being secured to said blood collection bag by said strap.

14. A blood preservation method, the method comprising:
collecting stem cells in a blood preservation apparatus, said apparatus having a plurality of blood collection containers being separated by at least one separator mechanism that enables rapid separation of at least one said blood collection container from said blood preservation apparatus;
freezing said blood preservation apparatus at a temperature suitable for preservation of the contents stored within said blood preservation apparatus; and
separating at least one blood collection container from said blood preservation apparatus while non-required elements of said blood preservation apparatus remain substantially frozen.

15. The method of claim 14, wherein said blood collection container is selected from the group consisting of blood collection bags and blood entry tubes.

16. The method of claim 14, comprising separating at least a portion of a blood collection container from said blood preservation apparatus while non-required blood collection containers in said blood preservation apparatus remain substantially frozen.

17. The method of claim 14, comprising separating at least a portion of a selected size from said blood preservation apparatus while non-required elements of said blood preservation apparatus remain substantially frozen.

18. The method of claim 14, comprising:
extracting said blood preservation apparatus from a blood freezing source to remove at least one portion of said apparatus; and
returning said blood preservation apparatus to said blood freezing source before the contents of said blood preservation apparatus have substantially thawed.

19. The method of claim 14, comprising expanding the contents extracted from said separated portion.

20. The method of claim 14, wherein said separating includes removing at least a portion of said blood preservation apparatus using one or more of actions selected from the group consisting of guillotining said separator mechanism, tearing off said portion along a perforated line, tearing apart a hook and loop seal, unbuttoning a button, unzipping a zipping mechanism, untying a knot and separating an adhesive seal.

21. The method of claim 14, comprising heat sealing at least one portion of said blood preservation apparatus.
